(19) United States
(12) Patent Application Publication
(10) Pub. No.: US 2024/0053955 A1
(43) Pub. Date: Feb. 15, 2024

(54) HEARING MONITORING METHOD AND APPARATUS

(71) Applicant: HUAWEI TECHNOLOGIES CO., LTD., SHENZHEN, GUANGDONG (CN)

(72) Inventors: Chang LIU, Shenzhen (CN); Hui YANG, Beijing (CN)

(21) Appl. No.: 18/496,140

(22) Filed: Oct. 27, 2023

Related U.S. Application Data
(63) Continuation of application No. PCT/CN2021/091311, filed on Apr. 30, 2021.

(51) Int. Cl. G06F 3/16; A61B 5/12; A61B 5/117

(52) U.S. Cl. CPC G06F 3/165; A61B 5/125; A61B 5/117

(57) ABSTRACT

This application provides a hearing monitoring method and apparatus. The method includes: obtaining ear overuse information of a user, where the ear overuse information includes accumulated duration and/or accumulated times of overuse of ears of the user; and generating prompt information or a control instruction based on the ear overuse information. In this solution, because accumulated information is obtained, impact of a misoperation of the user can be excluded, and continued overuse of ears is also fully detected. Therefore, an unnecessary or missed reminder is avoided, thereby achieving more accurate hearing monitoring effect.

### CROSS-REFERENCE TO RELATED APPLICATIONS

This application is a continuation of International Application No. PCT/CN2021/091311, filed on Apr. 30, 2021, the disclosure of which is hereby incorporated by reference in its entirety.

### TECHNICAL FIELD

This application relates to the field of hearing, and more specifically, to a hearing monitoring method and apparatus.

### BACKGROUND

Many multimedia devices such as a TV, a stereo, a headset, and a computer enrich people's lives, but may bring some harm to people's hearing. Most young people do not work in “high-risk” industries where noise is close to them, but they have experienced a significant decline in hearing (that is, hearing loss). A most direct cause of hearing loss is overuse of ears, and a measurement criterion of “overuse” is mainly indicated in two aspects: excessively high volume and excessively long duration.

To avoid hearing loss, some monitoring solutions are provided. For example, to avoid an excessively high volume of a headset, when it is detected that a volume exceeds a threshold, a user is reminded or the volume is controlled to be lowered. For another example, to avoid excessively long audio listening duration, when it is detected that duration exceeds a threshold, the user is reminded or audio is turned off. However, in an existing solution, only single audio listening volume or duration of the user is monitored. This is equivalent to that the user is reminded to protect hearing before single overuse of ears occurs. However, sometimes the user may just accidentally turn the volume up and quickly adjust it back. In this case, a reminder is unnecessary. Sometimes, many people do not care about the single overuse of ears, because most people subconsciously think that overuse of ears for a single time or two times does not matter. “Accidental” overuse of ears that these users think is a real reason of hearing damage, but the users do not realize that, and the conventional technology also ignores this case.

In short, an existing single monitoring solution does not take into account cases such as a misoperation of the user or continued overuse of ears of the user, resulting in an inaccurate monitoring result. Therefore, how to monitor hearing more accurately is a technical problem that needs to be urgently resolved.

### SUMMARY

This application provides a hearing monitoring method and apparatus, to monitor hearing more accurately.

According to a first aspect, a hearing monitoring method is provided. The method includes: obtaining ear overuse information of a user, where the ear overuse information includes accumulated duration and/or accumulated times of overuse of ears of the user; and generating prompt information or a control instruction based on the ear overuse information.

In technical solutions of this application, because accumulated information is obtained, impact of a misoperation of the user can be excluded, and continued overuse of ears is also fully detected. Therefore, an unnecessary or missed reminder is avoided, thereby achieving more accurate hearing monitoring effect.

As described above, overuse of ears may be understood as ear use in an unhealthy case, or ear use in a case that may cause damage. Ear use may be understood as listening to audio, listening to a sound emitted by a sound-producing device, listening to a sound in an environment, and the like. Ears may directly obtain the sound from the environment, or obtain a sound emitted by a playing device by using the sound-producing device close to the ears, such as a headset.

A most intuitive manifestation of overuse of ears is that a heard sound volume is excessively high (excessively high volume) and continuous listening duration is excessively long (excessively long duration).

Whether the sound is obtained from the environment or the sound is obtained by using the sound-producing device close to the ears such as the headset, a threshold may be properly set according to some health principles. When the sound volume is greater than or equal to the threshold, it may be considered as overuse of ears. For example, a volume threshold or a use time threshold of the sound-producing device (whether the sound-producing device is close to the ears or not) may be set.

With reference to the first aspect, in some embodiments, the ear overuse information may specifically include accumulated duration and accumulated times that the user listens to audio with a volume greater than or equal to a volume threshold, that is, accumulated duration and accumulated times of the excessively high volume in overuse of ears.

With reference to the first aspect, in some embodiments, the ear overuse information may specifically include accumulated duration in which audio listening duration of the user is greater than or equal to a single duration threshold, that is, accumulated duration of the excessively long duration in overuse of ears. It is assumed that the single duration threshold is 60 minutes. The accumulated duration is equal to accumulated duration that the user continuously listens to the audio for more than 60 minutes each time.

The prompt information or the control instruction may be used to remind the user or control a volume or a switch of an audio device (including the sound-producing device and the playing device). For example, the prompt information or the control instruction may be reminding the user to lower the volume or turn off the audio, or generating a control signal to control the volume of the sound-producing device or the playing device or turn off the played audio.

The prompt information may include at least one of the following: the accumulated duration, the accumulated times, single maximum duration of listening to the audio over time, a single maximum volume of listening to audio with the excessively high volume, or information reminding to lower the volume or turn off the audio. The control instruction may include an instruction for lowering the volume or an instruction for turning off the audio.

With reference to the first aspect, in some embodiments, a hearing level may further be introduced, and monitoring of the hearing level may further be introduced. In other words, the prompt information or the control instruction may alternatively be obtained based on the hearing level, and the hearing level may be obtained based on a bioelectrical signal of the user. The bioelectrical signal may include at least one of the following: an electroencephalogram signal, an electrocardiogram signal, a heart rate signal, and the like. In this embodiment of this application, a bioelectrical signal that can be used to evaluate a hearing status of the user is used. Machine learning methods such as a deep learning method, a neural network method, and a support vector machine may be used to test the listening level. For example, a hearing level model may be obtained through training. The model uses the bioelectrical signal as an input, and the hearing level as an output.

In an embodiment, the hearing level of the user may be obtained. The hearing level is obtained based on the bioelectrical signal of the user, and the prompt information or the control instruction is obtained based on the ear overuse information and the hearing level.

With reference to the first aspect, in some embodiments, the prompt information or the control instruction may be sent when a trigger condition is met. The trigger condition may include at least one of the following: Interval duration is greater than or equal to a sending period, the accumulated duration is greater than or equal to a duration threshold, a quantity of times of overuse of ears is greater than or equal to a quantity threshold, and the hearing level decreases. For example, if the sending period is one month, the duration threshold is two hours, and the quantity threshold is five, it is equivalent to sending the prompt information or the control instruction every month, or when the accumulated duration of overuse of ears is greater than or equal to two hours, or when the quantity of times of overuse of ears is greater than or equal to five. These preset conditions may be set separately or may be set in a superposition mode. This is not limited.

The sent prompt information may have any one or more of the following presentation manners: a message, voice, audio, animation, and the like. The sent control instruction may be a control instruction for lowering the volume or turning off the audio.

In an embodiment, the trigger condition may be fixedly set, or may be adjusted by the user.

With reference to the first aspect, in some embodiments, when the prompt information or the control instruction is sent, the prompt information or the control instruction may be sent to the user based on a user identifier. In other words, the prompt information or the control instruction is sent to the current user only when the user identifier is consistent with the current user. In this way, data security of the user can be ensured, matched data can be sent to the user, and a plurality of users can share a same hearing monitoring apparatus. It may be understood that the prompt information or the control instruction is sent to the user based on the user identifier of the user.

In an embodiment, the user identifier may be obtained by using technologies such as a bioelectrical signal, a user identity, facial recognition, voiceprint recognition, and fingerprint recognition. It may also be understood that the user identifier is obtained by using at least one of the following: the bioelectrical signal, the user ID, face information, voiceprint information, and fingerprint information.

According to a second aspect, a hearing monitoring apparatus is provided. The apparatus includes a unit that can implement the method in any one of the first aspect or the embodiments of the first aspect.

According to a third aspect, a non-transitory, computer-readable storage medium is provided. The computer-readable storage medium stores computer instructions. When the computer instructions are run on a computer, the computer is enabled to perform the method in any one of the first aspect or the embodiments of the first aspect.

According to a fourth aspect, a computer program product is provided. The computer program product includes computer program code. When the computer program code is run on a computer, the computer is enabled to perform the method in any one of the first aspect or the embodiments of the first aspect.

### BRIEF DESCRIPTION OF DRAWINGS

FIG. 1 is a schematic flowchart of a hearing monitoring method according to an embodiment of this application;

FIG. 2 is a schematic flowchart of a method for obtaining a single overuse of ears case according to an embodiment of this application;

FIG. 3 is a schematic flowchart of a method for obtaining a single overuse of ears case according to an embodiment of this application;

FIG. 4 is a schematic diagram of a hearing monitoring apparatus according to an embodiment of this application; and

FIG. 5 is a schematic diagram of a hardware structure of a hearing monitoring apparatus according to an embodiment of this application.

### DETAILED DESCRIPTION

The following describes technical solutions of this application with reference to accompanying drawings.

To facilitate understanding of the technical solutions of this application, the following describes technical terms and concepts in this application.

(1) Bioelectrical Signal

Bioelectricity refers to a regular electrical phenomenon that is closely related to a life status, which is produced by active cells or tissues (a human body or animal tissues) whether in a static or active state.

A bioelectrical signal includes a resting potential and an action potential, and essence of the bioelectrical signal is an across-membrane flow of ions. Common bioelectrical signals are electromyogram, electroencephalogram, electrocardiogram, electrooculogram, heart rate, and so on. These bioelectrical signals, which can be measured on a body surface, are also referred to as body surface bioelectrical signals. Because bioelectrical signals such as electromyogram, electroencephalogram, electrocardiogram, and electrooculogram have specific differences in frequency bands, amplitudes, waveforms, and the like, one or more bioelectrical signals may be obtained by processing the bioelectrical signals collected by an electrode. In the conventional technology, these bioelectrical signals are usually recorded as electromyogram, electroencephalogram, electrocardiogram, and the like, to provide help for related professionals to perform scientific researches and medical diagnosis. In embodiments of this application, a signal that can reflect a case when a user listens to audio, such as the electroencephalogram, the electrocardiogram, or the heart rate, is mainly used to perform a hearing test.

(2) Electroencephalogram Signal

An electroencephalogram (EEG) signal, also referred to as a brain wave signal, is an external manifestation of a brain activity, and different brain activities manifest as electroencephalogram signals with different characteristics. Research shows that time-space-frequency domain analysis of these electroencephalogram patterns is helpful for reverse analysis of human intentional activities. This provides a theoretical basis for application of the electroencephalogram signal.

A scalp electroencephalogram signal is a kind of bioelectrical signal. An electric field formed by potential changes of 86 billion neurons in a brain is conducted through a volume conductor formed by a cortex, a skull, meninges and a scalp to generate potential distribution on the scalp. The electroencephalogram signal can be obtained by recording changing potential distribution with a specific device.

The electroencephalogram signal may be divided into spontaneous electroencephalogram and evoked (EP) electroencephalogram. The spontaneous electroencephalogram is a potential change of nerve cells in the brain without specific external stimulation. The evoked electroencephalogram is a brain potential change caused by different types of stimulation of nerve cells in the brain, such as sound, light, and electricity. A hearing status of a person can be obtained by detecting an electroencephalogram signal of the person.

As known above, a single monitoring solution in the conventional technology cannot accurately monitor hearing of the user. A main reason is that some cases are missed, such as the foregoing case where the user just performs a short-term misoperation, and the foregoing case where the user forcibly uses ears excessively (in other words, a case where the user ignores a single monitoring result and continues to listen to the audio with an excessively high volume or over time). For a life-oriented example, in the conventional technology, when an audio volume exceeds a threshold or continuous audio listening duration exceeds a threshold, the user is reminded, but if the user just performs a misoperation or continues to use the ears excessively, the case cannot be detected. That is, there is an unnecessary or missed reminder.

For the foregoing problem, embodiments of this application provide a hearing monitoring method. Accumulated duration and/or accumulated times of overuse of ears of the user are obtained, and prompt information or a control instruction is generated based on the information, to achieve more accurate hearing monitoring effect. Because accumulated information is obtained, impact of the misoperation can be excluded, and continued overuse of ears is also fully detected. Therefore, the unnecessary or missed reminder is avoided.

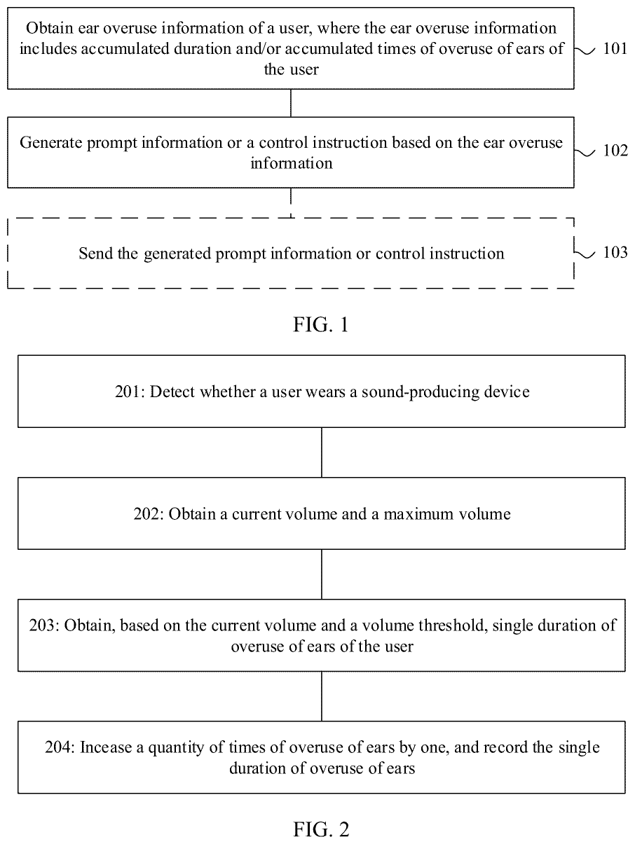

FIG. 1 is a schematic flowchart of a hearing monitoring method according to an embodiment of this application. The following describes operations shown in FIG. 1.

101: Obtain ear overuse information of a user, where the ear overuse information includes accumulated duration and/or accumulated times of overuse of ears of the user.

As described above, overuse of ears may be understood as ear use in an unhealthy case, or ear use in a case that may cause damage. Ear use may be understood as listening to audio, listening to a sound emitted by a sound-producing device, listening to a sound in an environment, and the like. Ears may directly obtain the sound from the environment, or obtain a sound emitted by a playing device by using the sound-producing device close to the ears, such as a headset.

In this embodiment of this application, the sound-producing device may include, for example, the headset, a microphone, smart glasses, a mobile phone, a tablet, a computer, an audio device such as a sound box, a vehicle-mounted audio and video device, and a loudspeaker. The playing device may include a mobile phone, a tablet, a computer, an audio device such as a sound box, a vehicle-mounted audio and video device, and the like. In other words, the sound-producing device is a device that produces a sound, and the playing device is a device that can play audio or video in the sound-producing device. Therefore, there are two cases. One is that the playing device directly broadcasts the audio to the environment, and human ears obtain the sound from the environment. This may be called “public playing” or “external playing”. Because the audio sound can be heard by any person in the environment, the playing device is the sound-producing device. For example, when people watch TV at home and watch movies in a theater, the playing device is the TV and a movie player. The other is that the playing device needs to use another sound-producing device to transmit a sound to a person. For example, when a computer is connected to a stereo or a headset, the playing device is the computer, and the sound-producing device is the stereo or the headset. When the sound-producing device is the stereo, it is still in a form of “public playing”. When the sound-producing device is the headset, only a person wearing the headset can hear the audio, because a sound played by the playing device is so little transmitted to the environment in which other people cannot hear the sound.

A most intuitive manifestation of overuse of ears is that a heard sound volume is excessively high (excessively high volume) and continuous listening duration is excessively long (excessively long duration).

For the sound-producing device used close to the ears, such as the headset and the smart glasses, a volume and use time of the sound-producing device can be controlled, to use the ears healthily. An internationally recognized principle of headset use for hearing protection is called “60-60” principle, which means that when a headset is used, a volume should not exceed 60% of a maximum volume, and it is best to adjust the volume to a lower level; and continuous headset use duration should not exceed 60 minutes. Adults should not wear headsets for more than 3 to 4 hours a day, minors should not wear headsets for more than 2 hours. In addition, after wearing headsets for 30 to 40 minutes each time, ears should be fully rested. When the above recommended value is exceeded, it can be considered as a manifestation of overuse of ears.

For obtaining the sound from the environment, for example, using a TV, a tablet, or a mobile phone to watch a video or listen to audio in a public manner, the sound can be obtained from the environment. In this case, whether it is overuse of ears can be determined according to a noise principle. In other words, sound exceeding a specific decibel is noise, which is not appropriate. It is also not appropriate to watch video or listen to audio for a long time, especially in a noisy environment, which causes greater damage. However, it should be understood that for obtaining the sound of the playing device from the environment, a playing volume of the playing device may be limited to control the volume and duration of the sound that the user obtains from the environment when using the playing device. To be specific, a manner similar to that of the headset is used, for example, a volume threshold of the playing device is set to 60% or 50% of a maximum volume of the playing device.

In other words, whether the sound is obtained from the environment or the sound is obtained by using the sound-producing device close to the ears such as the headset, a threshold may be properly set according to some health principles. When the sound volume is greater than or equal to the threshold, it may be considered as overuse of ears. For example, a volume threshold or a use time threshold (that is, a single duration threshold described below) of the sound-producing device (whether the sound-producing device is close to the ear or not) may be set.

In some embodiments, the ear overuse information may specifically include accumulated duration and accumulated times that the user listens to audio with a volume greater than or equal to a volume threshold, that is, accumulated duration and accumulated times of the excessively high volume in overuse of ears.

In other embodiments, the ear overuse information may specifically include accumulated duration in which audio listening duration of the user is greater than or equal to the single duration threshold, that is, accumulated duration of the excessively long duration in overuse of ears. It is assumed that the single duration threshold is 60 minutes. The accumulated duration is equal to accumulated duration that the user continuously listens to the audio for more than 60 minutes each time.

102: Generate prompt information or a control instruction based on the ear overuse information.

The prompt information or the control instruction may be respectively used to remind the user or control a volume or a switch of an audio device (including the sound-producing device and the playing device). For example, the prompt information or the control instruction may be reminding the user to lower the volume or turn off the audio, or generating a control signal to control the volume of the sound-producing device or the playing device or turn off the played audio.

The prompt information may include at least one of the following: the accumulated duration, the accumulated times, single maximum duration of listening to the audio over time, a single maximum volume of listening to audio with the excessively high volume, or information reminding to lower the volume or turn off the audio. The control instruction may include an instruction for lowering the volume or an instruction for turning off the audio.

103: Send the prompt information or the control instruction.

It should be noted that operation 103 may or may not be performed.

In an embodiment, the prompt information or the control instruction may be sent when a trigger condition is met. The trigger condition may include at least one of the following: Interval duration is greater than or equal to a sending period (in other words, periodic sending), the accumulated duration of overuse of ears is greater than or equal to a duration threshold, a quantity of times of overuse of ears is greater than or equal to a quantity threshold, and a hearing level decreases. For example, if the sending period is one month, the duration threshold is two hours, and the quantity threshold is five, it is equivalent to sending the prompt information or the control instruction every month, or when the accumulated duration of overuse of ears is greater than or equal to two hours, or when the quantity of times of overuse of ears is greater than or equal to five. These preset conditions may be set separately or may be set in a superposition mode. This is not limited.

The sent prompt information may have any one or more of the following presentation manners: a message, voice, audio, animation, and the like. The sent control instruction may be a control instruction for lowering the volume or turning off the audio.

It should be noted that the interval duration may be understood as a time interval formed by natural time. In other words, after a time start point is set, a time interval from the time start point is the interval duration. For example, the sending the prompt information or the control instruction every month means that the prompt information or the control instruction is sent once every month in natural time. The prompt information or the control instruction may alternatively be sent every week, every quarter, every 50 days, and so on. The month, week, quarter, or 50 days is the sending period. For example, the time start point may be a delivery date, or may be running time of a device, or may be the New Year's Day of each year, or may be set by the user independently.

In an embodiment, the trigger condition may be fixedly set, or may be adjusted by the user.

According to the method shown in FIG. 1, the prompt information or the control instruction is generated mainly by obtaining the accumulated duration and/or the accumulated times of overuse of ears of the user. Compared with the conventional technology, the method can achieve long-term monitoring, and can effectively exclude a misoperation of the user because the accumulated duration caused by the misoperation is not enough to affect hearing monitoring. In addition, “accidental” overuse of ears that is ignored by the user can be effectively monitored. In this way, hearing can be monitored more accurately.

In some embodiments, monitoring of the hearing level may further be introduced. In other words, the prompt information or the control instruction may alternatively be obtained based on the hearing level, and the hearing level may be obtained based on a bioelectrical signal of the user. The bioelectrical signal may include at least one of the following: an electroencephalogram signal, an electrocardiogram signal, a heart rate signal, and the like. In this embodiment of this application, a bioelectrical signal that can be used to evaluate a hearing status of the user is used. Machine learning methods such as a deep learning method, a neural network method, and a support vector machine may be used to test the listening level. For example, a hearing level model may be obtained through training. The model uses the bioelectrical signal as an input, and the hearing level as an output.

In an embodiment, the hearing level of the user may be obtained. The hearing level is obtained based on the bioelectrical signal of the user, and the prompt information or the control instruction is obtained based on the ear overuse information and the hearing level.

In some embodiments, user identification may further be introduced. In other words, when operation 103 is performed, the prompt information or the control instruction is sent to the user based on the user identifier. In other words, the prompt information or the control instruction is sent to the current user only when the user identifier is consistent with the current user. In this way, data security of the user can be ensured, matched data can be sent to the user, and a plurality of users can share a same hearing monitoring apparatus. It may be understood that the prompt information or the control instruction is sent to the user based on the user identifier of the user.

In an embodiment, the user identifier may be obtained by using technologies such as a bioelectrical signal, a user identity (ID), facial recognition, voiceprint recognition, and fingerprint recognition. It may also be understood that the user identifier is obtained by using at least one of the following: the bioelectrical signal, the user ID, face information, voiceprint information, and fingerprint information.

FIG. 2 is a schematic flowchart of a method for obtaining a single overuse of ears case according to an embodiment of this application. The following describes operations in FIG. 2.

201: Detect whether a user wears a sound-producing device.

In an embodiment, in a detection method, whether the user wears the sound-producing device may be determined by analyzing data obtained by using a sensor or the like. The sensor may be any one or more of the following: a distance sensor, a temperature sensor, and a bioelectrical signal sensor. Several sensors are separately explained below.

When the distance sensor is used, if the user wears the sound-producing device, a distance between the user and the sound-producing device is very close. Therefore, when an actual measured distance is less than or equal to a distance threshold, it may be considered that the user has worn the sound-producing device.

When the temperature sensor is used, if the user wears the sound-producing device, a temperature collected by the sound-producing device is a temperature of a human body surface. Therefore, when an actual measured temperature is within a human body temperature range, it may be considered that the user has worn the sound-producing device.

When the bioelectrical signal sensor is used, if the user wears the sound-producing device, the sound-producing device can collect a bioelectrical signal. Therefore, when the bioelectrical signal can be collected, it may be considered that the user has worn the sound-producing device.

It should be noted that the sound-producing device herein may be a near-ear device such as a headset or smart glasses, in other words, the user needs to obtain a sound by using the worn sound-producing device. If the user directly obtains the sound from an environment, operation 201 does not need to be performed.

In an embodiment, if a detection result of operation 201 is that the user has worn the sound-producing device, subsequent operations are performed. If the detection result is that the user does not wear the sound-producing device, operation 201 may be repeatedly performed.

202: Obtain a current volume and a maximum volume.

The current volume refers to an audio volume obtained by the user from the sound-producing device currently. It is assumed that the user is listening to music played by a mobile phone by using the headset, the current volume is an audio volume of the mobile phone. The maximum volume refers to a maximum volume of audio obtained by the user from the sound-producing device currently without considering hearing health. It is assumed that the user is listening to music played by the mobile phone by using the headset, the current volume is a maximum volume supported by the mobile phone. The maximum volume may be used to determine a volume threshold. For example, the volume threshold may be set to x times of the maximum volume, where x is a real number greater than 0 and less than or equal to 1. In this case, the volume threshold can be obtained as long as the maximum volume is known, in other words, the volume threshold may vary based on a maximum volume of the sound-producing device and/or a playing device. However, it should be understood that the volume threshold may alternatively be a fixed value. In this case, the maximum volume does not need to be obtained.

It should be noted that the current volume and the maximum volume may be determined by at least one of the sound-producing device and the playing device. For example, it is assumed that the user uses a listening headset (a headset that is commonly used in listening exams in English classes and that can listen to the radio) to listen to audio, the sound-producing device is the playing device, the current volume is a playing volume of the listening headset, and the maximum volume is a maximum volume of the listening headset. For another example, it is assumed that the user listens to audio played by a notebook computer by using a bone conduction headset, the bone conduction headset is connected to the notebook computer through Bluetooth, and the bone conduction headset has its own volume setting. In this case, more accurate explanation of the current volume and the maximum volume is explanation in content of a previous paragraph. The current volume and the maximum volume may be jointly determined by two parts: a volume of the bone conduction headset and a volume of the notebook computer, and may alternatively be determined by three parts: the volume of the bone conduction headset, a system volume of the notebook computer, and a volume of audio playing software on the notebook computer. For another example, it is assumed that the user listens to music played by a mobile phone by using a common wired headset. In this case, the current volume and the maximum volume are determined by the playing device (which is the mobile phone herein), the sound-producing device (which is the headset herein), the current volume is a playing volume of the mobile phone, and the maximum volume is a maximum volume of the mobile phone.

203: Obtain, based on the current volume and the volume threshold, single duration of overuse of ears of the user.

It should be noted that overuse of ears of the user in operation 203 mainly refers to an excessively high volume in overuse of ears. Therefore, the single duration may be obtained based on a relationship between the current volume and the volume threshold. When the current volume is greater than or equal to the volume threshold, it is considered that overuse of ears occurs. The single duration of overuse of ears herein refers to single duration for which user listens audio with a volume greater than or equal to the volume threshold.

In some embodiments, the single duration may be obtained by timing a time period in which “the current volume is greater than or equal to the volume threshold”. For example, when the current volume is greater than or equal to the volume threshold, a timer is triggered to start timing. In a timing process, operation 201 and operation 202 may still be performed, and then timing is stopped when a timing stop condition is met. In this way, the single duration can be obtained. The timing stop condition may be, for example, any one of the following: The user lowers the volume to be less than the volume threshold, the audio stops, or the user removes the sound-producing device.

Several timing stop conditions are explained below.

The current volume is continuously obtained over time. Whenever a volume changes, a changed volume may be compared with the volume threshold. When the volume is less than the volume threshold (in other words, the user lowers the volume to be less than the volume threshold), timing is stopped and single duration is obtained.

That the audio stops means that the playing device stops playing audio or the sound-producing device stops producing a sound. This case may be considered as a special case in which the volume is less than the volume threshold, which is equivalent to that the volume is 0, or may be considered as an example in which the user stops listening to the audio.

If the user removes the sound-producing device, it is equivalent to that a user wearing status changes. When it is detected that the user does not wear the sound-producing device, timing is stopped, and single duration is obtained. This case may also be considered as an example that the user stops listening to the audio.

It can be learned from the foregoing explanation that the user stops listening to the audio may also be considered as a timing stop condition, and the timing stop condition may further include two cases: The audio stops and the user removes the sound-producing device.

To make the timing more accurate, the timer may further be cleared before the timer starts timing. Alternatively, the timer may be cleared after each timing ends, to facilitate subsequent timing.

For example, the volume threshold may be set to 60% of the maximum volume, and may alternatively be set to 80 decibels if it is in a form of external playing. This is not enumerated.

204: Increase a quantity of times of overuse of ears by one, and record the single duration of overuse of ears.

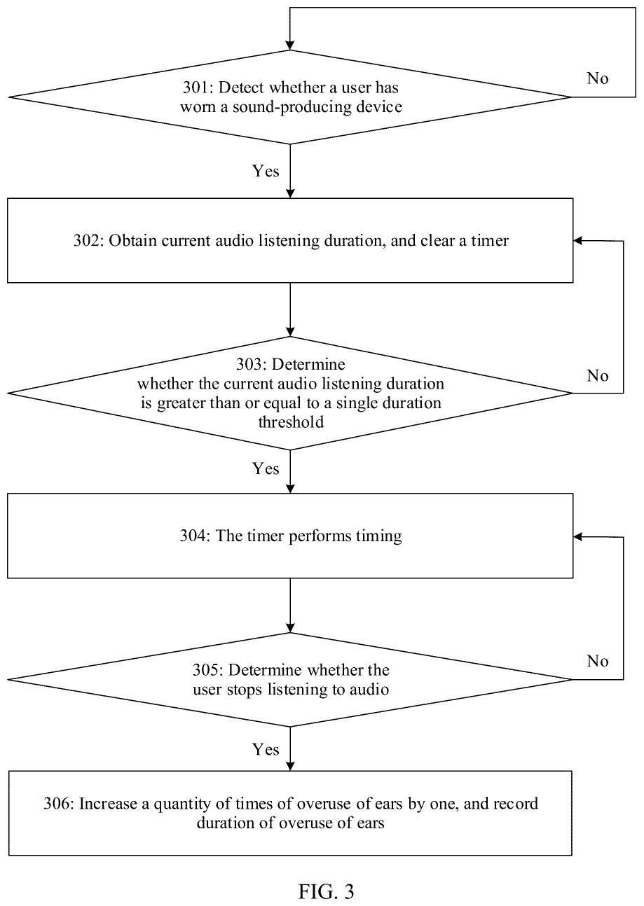

The single overuse of ears case may be obtained by using the method shown in FIG. 2. If the operations shown in FIG. 2 are repeatedly performed, a plurality of single overuse of ears cases may be obtained, and the accumulated duration and the accumulated times of overuse of ears in the ear overuse information in operation 101 can be obtained. However, it should be understood that FIG. 2 shows only a case in which the volume is excessively high (excessively high volume) in overuse of ears, and a case in which audio listening duration is excessively long is described below with reference to FIG. 3.

FIG. 3 is a schematic flowchart of a method for obtaining a single overuse of ears case according to an embodiment of this application. The following describes operations in FIG. 3.

301: Detect whether a user has worn a sound-producing device. When a result is “yes”, operation 301 is performed; and when the result is “no”, operation 301 is performed.

For operation 301, refer to the description of operation 201. For brevity, details are not described again.

302: Obtain current audio listening duration, and clear a timer.

Operation 302 is mainly to obtain duration in which the user has listened to audio at a current moment, for example, 10 minutes, 1 hour, or 2.5 hours. Clearing the timer is to prepare for subsequent timing.

303: Determine whether the current audio listening duration is greater than or equal to a single duration threshold. When a determining result is “yes”, operation 304 is performed; and when the determining result is “no”, operation 302 is performed.

304: The timer performs timing.

A combination of operation 303 and operation 304 is mainly to obtain duration in which the user continues to listen to the audio after the single duration threshold is exceeded, that is, another case of overuse of ears: excessively long audio listening duration. Duration of “excessively long” is obtained herein. The single duration threshold refers to a duration threshold for the user to listen to the audio each time.

305: Determine whether the user stops listening to the audio. When a determining result is “yes”, operation 306 is performed; and when the determining result is “no”, operation 304 is performed.

Operation 305 mainly provides an end moment of single timing, and this may be understood as a timing stop condition in operation 203. In other words, when the user stops listening to the audio, operation 306 is performed, and if the user does not stop listening to the audio, timing continues.

As described above, the user stops listening to the audio may be that the audio stops, and may alternatively be that the user removes the sound-producing device.

306: Increase a quantity of times of overuse of ears by one, and record single duration of overuse of ears.

The single duration of overuse of ears obtained in operation 306 is the duration in which the user continues to listen to the audio after the single duration threshold is exceeded. It is assumed that the single duration threshold is 65 minutes, and the user obtains, through the foregoing process, that timing duration of the timer is 30 minutes after the timer reaches 65 minutes. In this case, the single duration of overuse of ears is 30 minutes, and total continuous audio listening duration of the user is 95 minutes.

It should be noted that FIG. 2 and FIG. 3 separately show how to obtain the single overuse of ears case in two cases of overuse of ears (the excessively high volume and the excessively long duration). A plurality of single overuse of ears cases may be obtained by repeatedly performing the operations in FIG. 2 and FIG. 3, to obtain the accumulated duration and the accumulated times of overuse of ears in the ear overuse information in operation 1.

FIG. 4 is a schematic diagram of a hearing monitoring apparatus according to an embodiment of this application. As shown in FIG. 4, an apparatus 1000 includes an obtaining unit 1001 and a processing unit 1002, which may be configured to perform the hearing monitoring method in embodiments of this application.

For example, the obtaining unit 1001 may perform the foregoing operation 101, and the processing unit 1002 may perform the foregoing operation 102. When performing operation 101, the obtaining unit 1001 may be further configured to perform operations shown in FIG. 2 and FIG. 3, to obtain a single overuse of ears case, and obtain the ear overuse information in operation 101 based on a plurality of obtained single overuse of ears cases.

The obtaining unit 1001 and the processing unit 1002 may further perform the foregoing operation of monitoring a hearing level. The obtaining unit 1001 may be configured to obtain a hearing level of a user, where the hearing level is obtained based on a bioelectrical signal of the user. The processing unit 1002 may be configured to obtain prompt information or a control instruction based on the hearing level and/or the ear overuse information.

In an embodiment, the apparatus 1000 may further include a sending unit 1003, and the sending unit 1003 may be configured to perform the foregoing operation 103.

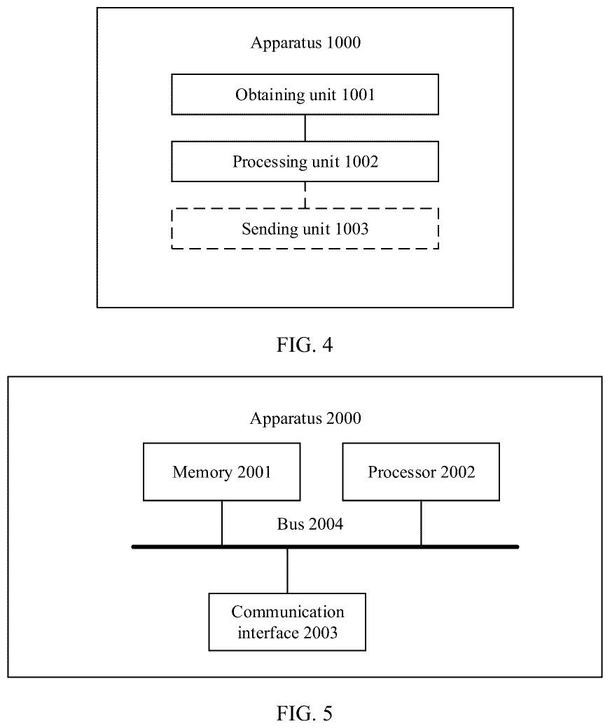

FIG. 5 is a schematic diagram of a hardware structure of a hearing monitoring apparatus according to an embodiment of this application. A hearing monitoring apparatus 2000 shown in FIG. 5 includes a memory 2001, a processor 2002, a communication interface 2003, and a bus 2004. Communication connections between the memory 2001, the processor 2002, and the communication interface 2003 are implemented through the bus 2004.

The memory 2001 may be a read-only memory (ROM), a static storage device, a dynamic storage device, or a random access memory (RAM). The memory 2001 may store a program. When the program stored in the memory 2001 is executed by the processor 2002, the processor 2002 and the communication interface 2003 are configured to perform operations of the hearing monitoring method in embodiments of this application.

The processor 2002 may be a general-purpose central processing unit (CPU), a microprocessor, an application-specific integrated circuit (ASIC), a graphics processing unit (GPU), or one or more integrated circuits, and is configured to execute a related program, to implement a function that needs to be performed by a unit of the hearing monitoring apparatus in this embodiment of this application, or perform the hearing monitoring method in the method embodiments of this application.

The processor 2002 may alternatively be an integrated circuit chip and has a signal processing capability. In an embodiment, the operations of the hearing monitoring method in embodiments of this application may be completed by using a hardware integrated logic circuit or an instruction in a form of software in the processor 2002.

The processor 2002 may be a general-purpose processor, a digital signal processor (DSP), an ASIC, a field programmable gate array (FPGA) or another programmable logic device, a discrete gate or transistor logic device, or a discrete hardware component. The general-purpose processor may be a microprocessor, or the processor may alternatively be any conventional processor or the like. Operations of the method disclosed with reference to embodiments of this application may be directly executed and completed by using a hardware decoding processor, or may be executed and completed by using a combination of hardware and software modules in the decoding processor. The software module may be located in a mature storage medium in the art, such as a random access memory, a flash memory, a read-only memory, a programmable read-only memory, an electrically erasable programmable memory, or a register. The storage medium is located in the memory 2001. The processor 2002 reads information in the memory 2001, and implements, in combination with hardware of the processor 2002, the function that needs to be performed by the unit in the hearing monitoring apparatus in this embodiment of this application, or performs the hearing monitoring method in the method embodiment of this application.

The communication interface 2003 uses, for example but not limited to, a transceiver apparatus of a transceiver type, to implement communication between the apparatus 2000 and another device or a communication network. For example, ear overuse information may be obtained through the communication interface 2003.

The bus 2004 may include a path for transferring information between components (for example, the memory 2001, the processor 2002, and the communication interface 2003) of the apparatus 2000.

It should be noted that although only the memory, the processor, and the communication interface in the apparatus 2000 are illustrated, in an embodiment, a person skilled in the art should understand that the apparatus 2000 may further include another component necessary for proper running. In addition, based on a specific requirement, a person skilled in the art should understand that the apparatus 2000 may further include hardware components for implementing other additional functions. In addition, a person skilled in the art should understand that the apparatus 2000 and the apparatus 4000 may include only components necessary for implementing embodiments of this application, but not necessarily include all the components shown in FIG. 5.

This application further provides a computer-readable storage medium. The computer-readable storage medium stores computer instructions. When the computer instructions are run on a computer, the computer is enabled to perform operations and/or processing in the hearing monitoring method provided in this application.

This application further provides a computer program product. The computer program product includes computer program code. When the computer program code is run on a computer, the computer is enabled to perform operations and/or processing in the hearing monitoring method provided in this application.

All or some of the foregoing embodiments may be implemented by using software, hardware, firmware, or any combination thereof. When the software is used to implement embodiments, the foregoing embodiments may be implemented completely or partially in a form of a computer program product. The computer program product includes one or more computer instructions or computer programs. When the computer instructions or the computer programs are loaded and executed on a computer, the procedures or functions according to embodiments of this application are all or partially generated. The computer may be a general-purpose computer, a special-purpose computer, a computer network, or another programmable apparatus. The computer instructions may be stored in a computer-readable storage medium or may be transmitted from a computer-readable storage medium to another computer-readable storage medium. For example, the computer instructions may be transmitted from a website, computer, server, or data center to another website, computer, server, or data center in a wired (for example, infrared, radio, or microwave) manner. The computer-readable storage medium may be any usable medium accessible by the computer, or a data storage device, such as a server or a data center, integrating one or more usable media. The usable medium may be a magnetic medium (for example, a floppy disk, a hard disk, or a magnetic tape), an optical medium (for example, a DVD), or a semiconductor medium. The semiconductor medium may be a solid-state drive.

It should be understood that the term “and/or” in this specification describes only an association relationship between associated objects, and represents that three relationships may exist. For example, A and/or B may represent the following three cases: Only A exists, both A and B exist, and only B exists. A and B may be singular or plural. In addition, the character “/” in this specification usually indicates an “or” relationship between the associated objects, but may also indicate an “and/or” relationship. For details, refer to the context for understanding.

In this application, “at least one” means one or more, and “a plurality of” means two or more. “At least one of the following items (pieces)” or a similar expression thereof refers to any combination of these items, including any combination of singular items (pieces) or plural items (pieces). For example, at least one of a, b, or c may indicate: a, b, c, a-b, a-c, b-c, or a-b-c, where a, b, and c may be singular or plural.

It should be understood that sequence numbers of the foregoing processes do not mean execution sequences in various embodiments of this application. The execution sequences of the processes should be determined based on functions and internal logic of the processes, and should not be construed as any limitation on the embodiments of this application.

A person of ordinary skill in the art may be aware that, in combination with the examples described in embodiments disclosed in this specification, units and algorithm operations may be implemented by electronic hardware or a combination of computer software and electronic hardware. Whether the functions are performed by hardware or software depends on particular applications and design constraint conditions of the technical solutions. A person skilled in the art may use different methods to implement the described functions for each particular application, but it should not be considered that the implementation goes beyond the scope of this application.

It may be clearly understood by a person skilled in the art that, for the purpose of convenient and brief description, for a detailed working process of the foregoing system, apparatus, and unit, refer to a corresponding process in the foregoing method embodiments. Details are not described herein again.

In several embodiments provided in this application, it should be understood that the disclosed system, apparatus, and method may be implemented in other manners. For example, the described apparatus embodiment is merely an example. For example, division into the units is merely logical function division and may be other division in actual implementation. For example, a plurality of units or components may be combined or integrated into another system, or some features may be ignored or not performed. In addition, the displayed or discussed mutual couplings or direct couplings or communication connections may be implemented through some interfaces. The indirect couplings or communication connections between the apparatuses or units may be implemented in electronic, mechanical, or other forms.

The units described as separate parts may or may not be physically separate, and parts displayed as units may or may not be physical units, may be located in one position, or may be distributed on a plurality of network units. Some or all of the units may be selected based on actual requirements to achieve the objectives of the solutions of embodiments.

In addition, functional units in embodiments of this application may be integrated into one processing unit, each of the units may exist independently physically, or two or more units may be integrated into one unit.

When the functions are implemented in a form of a software functional unit and sold or used as an independent product, the functions may be stored in a computer-readable storage medium. Based on such an understanding, technical solutions of this application essentially, or the part contributing to the conventional technology, or some of the technical solutions may be implemented in a form of a software product. The computer software product is stored in a storage medium, and includes several instructions for instructing a computer device (which may be a personal computer, a server, or a network device) to perform all or some of the operations of the methods described in embodiments of this application. The foregoing storage medium includes any medium that can store program code, such as a USB flash drive, a removable hard disk, a ROM, a RAM, a magnetic disk, or an optical disc.

The foregoing descriptions are merely specific embodiments of this application, but are not intended to limit the protection scope of this application. Any variation or replacement readily figured out by a person skilled in the art within the technical scope disclosed in this application shall fall within the protection scope of this application. Therefore, the protection scope of this application shall be subject to the protection scope of the claims.

1. A method of hearing monitoring, comprising:
obtaining ear overuse information of a user, wherein the ear overuse information comprises at least one of: accumulated duration or accumulated times of overuse of ears of the user; and
generating prompt information or a control instruction based on the ear overuse information.

2. The method according to claim 1, wherein the ear overuse information comprises accumulated duration and accumulated times that the user listens to audio with a volume greater than or equal to a volume threshold.

3. The method according to claim 1, wherein the ear overuse information comprises accumulated duration in which audio listening duration of the user is greater than or equal to a duration threshold.

4. The method according to claim 1, wherein generating the prompt information or the control instruction comprises:
obtaining a hearing level of the user based on a bioelectrical signal of the user; and
generating the prompt information or the control instruction based on the ear overuse information and the hearing level.

5. The method according to claim 1, further comprising:
sending the prompt information or the control instruction when a trigger condition is met, wherein the trigger condition comprises at least one of: interval duration is greater than or equal to a sending period, the accumulated duration is greater than or equal to a duration threshold, a quantity of times of overuse of ears is greater than or equal to a quantity threshold, a decrease in hearing level.

6. The method according to claim 5, wherein sending the prompt information or the control instruction comprises: sending the prompt information or the control instruction to the user based on a user identifier.

7. A non-transitory, computer-readable storage medium storing computer instructions, which when executed on a computer, cause the computer to perform operations, the operations comprising:
obtaining ear overuse information of a user, wherein the ear overuse information comprises at least one of: accumulated duration or accumulated times of overuse of ears of the user; and
generating prompt information or a control instruction based on the ear overuse information.

8. The non-transitory, computer-readable storage medium according to claim 7, wherein the ear overuse information comprises accumulated duration and accumulated times that the user listens to audio with a volume greater than or equal to a volume threshold.

9. The non-transitory, computer-readable storage medium according to claim 7, wherein the ear overuse information comprises accumulated duration and accumulated times in which single audio listening duration of the user is greater than or equal to a duration threshold.

10. The non-transitory, computer-readable storage medium according to claim 7, wherein generating the prompt information or the control instruction comprises:
obtaining a hearing level of the user based on a bioelectrical signal of the user; and
generating the prompt information or the control instruction based on the ear overuse information and the hearing level.

11. The non-transitory, computer-readable storage medium according to claim 7, wherein the operations further comprise:
sending the prompt information or the control instruction when a trigger condition is met, wherein the trigger condition comprises at least one of: interval duration is greater than or equal to a sending period, the accumulated duration is greater than or equal to a duration threshold, a quantity of times of overuse of ears is greater than or equal to a quantity threshold, or a decrease in hearing level.

12. The non-transitory, computer-readable storage medium according to claim 11, wherein sending the prompt information or the control instruction comprises sending the prompt information or the control instruction to the user based on a user identifier.

13. A hearing monitoring apparatus, comprising:
a processor; and
a memory coupled to the processor to store instructions, which when executed by the processor, cause the hearing monitoring apparatus to perform operations, the operations comprising:
obtaining ear overuse information of a user, wherein the ear overuse information comprises at least one of: accumulated duration or accumulated times of overuse of ears of the user; and
generating prompt information or a control instruction based on the ear overuse information.

14. The hearing monitoring apparatus according to claim 13, wherein the ear overuse information comprises accumulated duration and accumulated times that the user listens to audio with a volume greater than or equal to a volume threshold.

15. The hearing monitoring apparatus according to claim 13, wherein the ear overuse information comprises accumulated duration in which audio listening duration of the user is greater than or equal to a duration threshold.

16. The hearing monitoring apparatus according to claim 13, wherein generating the prompt information or the control instruction comprises:
obtaining a hearing level of the user based on a bioelectrical signal of the user; and
generating the prompt information or the control instruction based on the ear overuse information and the hearing level.

17. The hearing monitoring apparatus according to claim 13, wherein the operations further comprise:
sending the prompt information or the control instruction when a trigger condition is met, wherein the trigger condition comprises at least one of: interval duration is greater than or equal to a sending period, the accumulated duration is greater than or equal to a duration threshold, a quantity of times of overuse of ears is greater than or equal to a quantity threshold, a decrease in hearing level.

18. The hearing monitoring apparatus according to claim 17, wherein sending the prompt information or the control instruction comprises: sending the prompt information or the control instruction to the user based on a user identifier.
